(19) United States
(12) Patent Application Publication
(10) Pub. No.: US 2018/0344487 A1
(43) Pub. Date: Dec. 6, 2018

(54) Padded Shirt for Mastectomy

(71) Applicant: Nicole Elisabeth Marie Mommers-McCaffrey, New Milford, CT (US)

(72) Inventor: Nicole Elisabeth Marie Mommers-McCaffrey, New Milford, CT (US)

(73) Assignee: Nicole Elisabeth Marie Mommers-McCaffrey, New Milford, CT (US)

(21) Appl. No.: 15/988,324

(22) Filed: May 24, 2018

Related U.S. Application Data
(60) Provisional application No. 62/512,293, filed on May 30, 2017.

(51) Int. Cl. A61F 2/52; A41B 1/08

(52) U.S. Cl. CPC A61F 2/52; A41B 1/08

(57) ABSTRACT

A garment with a single piece of enhanced and contoured foam padding with integral breast mound prostheses and incorporated pectoral and sternal padding for the bilateral mastectomy patient is disclosed. This said piece of enhanced padding is sewn into the said garment to restore a natural looking breast mound appearance in clothing while offering padded comfort over the sensitive pectoral and sternal areas caused by a mastectomy. This said padding within the said garment is intended to be sewn onto a cropped, middle layer of material within the said garment. This said middle layer of material with attached padding is then sewn between the inside of the front, inner layer and the inside of the back, inner layer of the said garment with mound prostheses facing the inside of the front layer of the said garment thus giving a natural breast mound appearance on the outside of the front layer of the said garment. The said garment with said enhanced padding is intended for the bilateral mastectomy patient but also available to anyone else desiring to have a natural breast mound appearance in clothing. This invention is unique in its design from anything currently available on the market today in that it offers a single, fully padded piece of lightweight, contoured and enhanced foam padding with two, integral breast mound prostheses which is sewn directly, appropriately and permanently into a garment. There is no shelf bra, no pocket, no broad band elastic, no constriction and no heaviness within this invention. This invention is extremely convenient, lightweight, machine washable and machine dryable thus making it an instant, comfortable and complementary solution for the desire to restore or gain a natural breast mound appearance in clothing. No other breast prostheses are needed to have a complete and natural looking breast mound appearance when wearing this invention. This invention is available in different sizes to accommodate all individuals.

### CROSS-REFERENCES

This application claims the benefit of U.S. Provisional Patent Application No. 62/512,293 filed on May 30, 2017, U.S. Provisional Patent No. 62/574,961 filed on Oct. 20, 2017, U.S. Provisional Patent No. 62/585,481 filed on Nov. 13, 2017, U.S. Provisional Patent No. 62/589,675 filed on Nov. 22, 2017 and U.S. Provisional Patent No. 62/622,094 filed on Jan. 25, 2018 which are hereby incorporated by reference in their entirety.

### BACKGROUND OF THE INVENTION

### Field of the Invention

This invention relates to convenient, lightweight breast prostheses that are placed in a garment for the mastectomy patient. More particularly, this invention relates to a quick, confident and convenient way to dress with breast prostheses that are permanently placed and sewn into a garment for the mastectomy patient. This said enhanced padding is intended to be sewn appropriately in position into fashionable garments thus creating an instant solution for the absence of breast mounds in clothes while having incorporated padding to help comfort and to help protect the sensitive pectoral and sternal areas caused by the mastectomy surgical procedure.

### Background

Research shows that many women have a psychological need for breast replacement after mastectomy, whether it is temporary or permanent. There are few options available today that are temporary, lightweight, flattering, comfortable, non-constricting and convenient. Women are typically offered one or more breast prostheses that are placed inside a breast pocket which is attached to the inside bodice of a garment. These garments can be heavy, unattractive, uncomfortable, constricting, inconvenient and expensive. This invention comes from the need and desire of wanting to restore a temporary breast mound appearance comfortably, safely, and conveniently after bilateral mastectomy while also wanting to protect the sensitive pectoral and sternal areas caused by mastectomy. Simple things like wearing a seatbelt, hugging people or playing with pets can be hurtful and uncomfortable to the sensitive mastectomy site. This invention helps to shield and protect that sensitive mastectomy site while offering lightweight breast prostheses in the garment. This invention comes from the need of women's garments with integral, permanent, lightweight breast prostheses that are safe, convenient, comfortable, machine washable, machine dryable and non-constricting. It is important for the mastectomy patient to not wear any constricting garments because of the increased risk of lymphedema after mastectomy and lymph node removal. Lymphedema is a condition of localized fluid retention in the body which can be worsened by ill-fitting elastic banded bras or garments. This garment invention rids the risk of any constriction when the padding invention component is sewn into the said garment appropriately and intentionally. There are mastectomy shirts on the market today but most are offered with pockets for a prosthesis, or are heavy, constrictive, not machine washable, not machine dryable, unflattering, expensive or just not comfortable. Also, none of the mastectomy garments on the market today have incorporated foam between two breast prostheses. This invention of a garment which permanently incorporates padding with two, integral breast mound prostheses is lightweight, comfortable, complementary, non-constricting, machine washable, machine dryable and more affordable than the average breast prosthesis. This invention will enable the wearer to simply dress with the said garment and have no need for additional breast prostheses or any breast prostheses adjustments. This invention intends to help restore the self-confidence and mental well being of a mastectomy patient or other person while being safe and comfortable. This invention is free of any elastic or heavy material. This invention is intended to offer a quick, lightweight, convenient and flattering breast mound appearance to the mastectomy patient or anyone else desiring to have an integral breast mound appearance in their garment.

### SUMMARY OF THE INVENTION

This present invention has resolved the above drawbacks of the current mastectomy wear on the market today by providing a convenient, comfortable, easily laundered and safe alternative to the many typical breast prostheses that are offered today. This said invention offers the wearer of the said garment an instant and appropriate breast mound appearance instead of needing two, separate breast prostheses. In addition, this invention offers the added protection between the breast mound prostheses area with its incorporated foam padding. The padding within the garment invention is permanent, fixed and complementary within the said garment. This invention is designed for the bilateral mastectomy patient but welcome to anyone to accommodate their need and/or desire of wanting to restore or gain a breast mound appearance in clothing. This invention will be available in many sizes and sewn into many different designs of garments such as a camisole with step-in ease for the post mastectomy patient, cropped camisoles, cropped tank tops, tank tops, crew neck shirts, v-neck shirts, t-shirts, dresses, sleepwear, underwear, bathing suits and more. The drawings of the invention are intended to show the essence of the invention. The invention will be available in many different sizes and with slight alterations of shape but the integrity and spirit of the invention will remain constant in that the said invention is a garment which incorporates one piece of foam padding with two, integral breast mound prostheses.

### BRIEF DESCRIPTION OF THE DRAWINGS

FIG. 1 is an illustration of one representation of the garment present invention;

FIG. 2 is a front perspective view of the padding component of the invention which will be sewn into the garment present invention thereof;

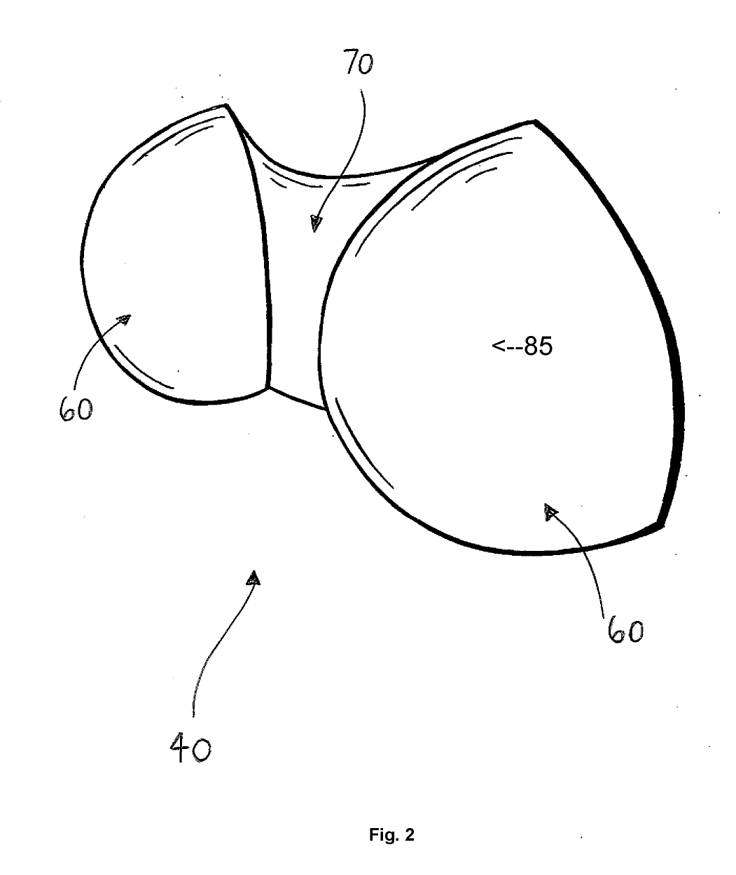

FIG. 3 is a front elevation view of the padding component of the invention which will be sewn into the garment present invention thereof;

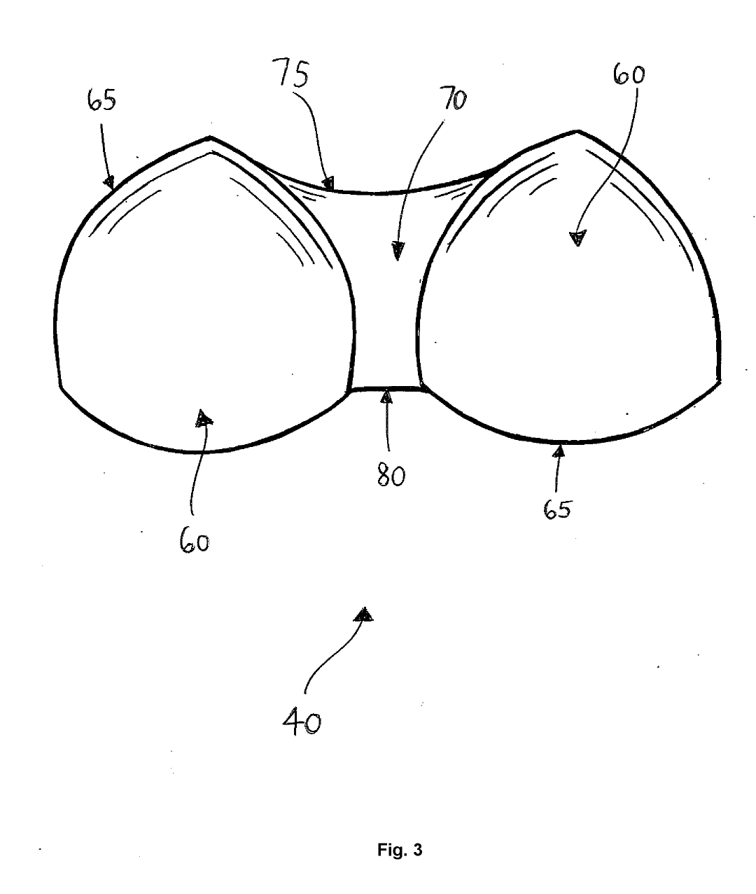

FIG. 4 is a rear perspective view of the padding component of the invention which will be sewn into the garment present invention thereof;

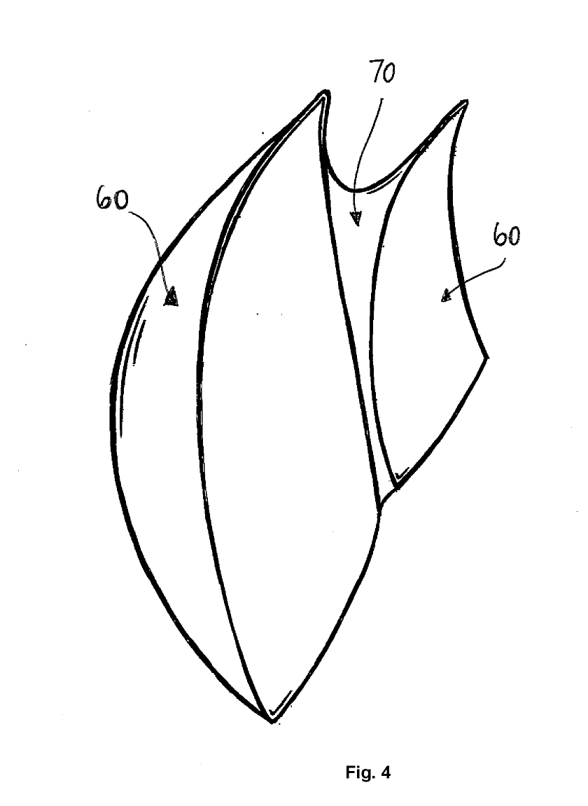

FIG. 5 is a side elevation view of the padding component of the invention which will be sewn into the garment present invention, the opposite being a mirror image thereof;

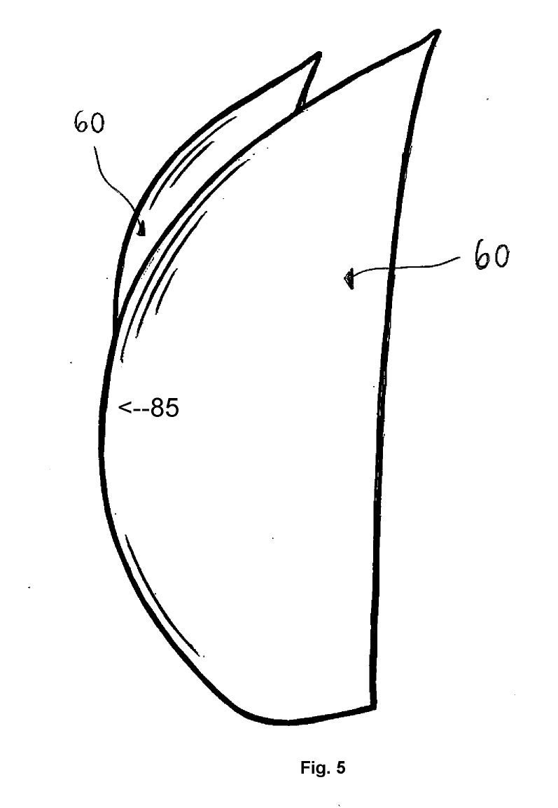

FIG. 6 is a bottom view of the padding component of the invention which will be sewn into the garment present invention thereof;

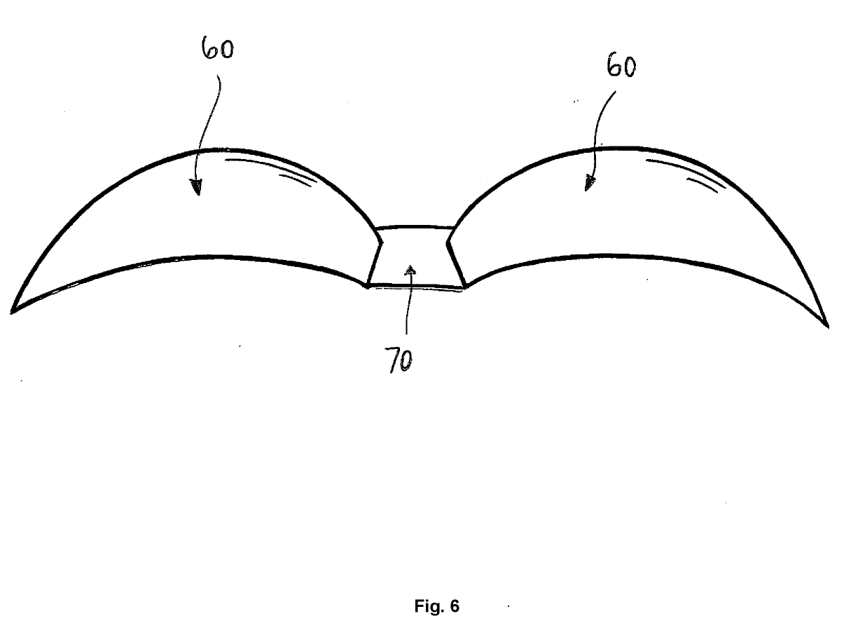

FIG. 7 is a cross-sectional view of the breast mound prosthesis component of the invention which will be sewn into the garment present invention thereof;

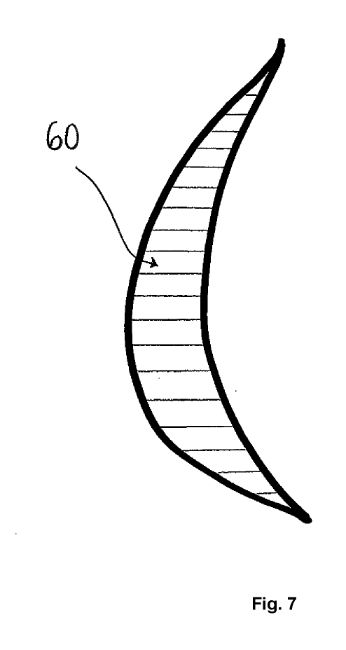

FIG. 8 is a front perspective view of a variation in shape of the padding component of the invention which will be sewn into the garment present invention thereof;

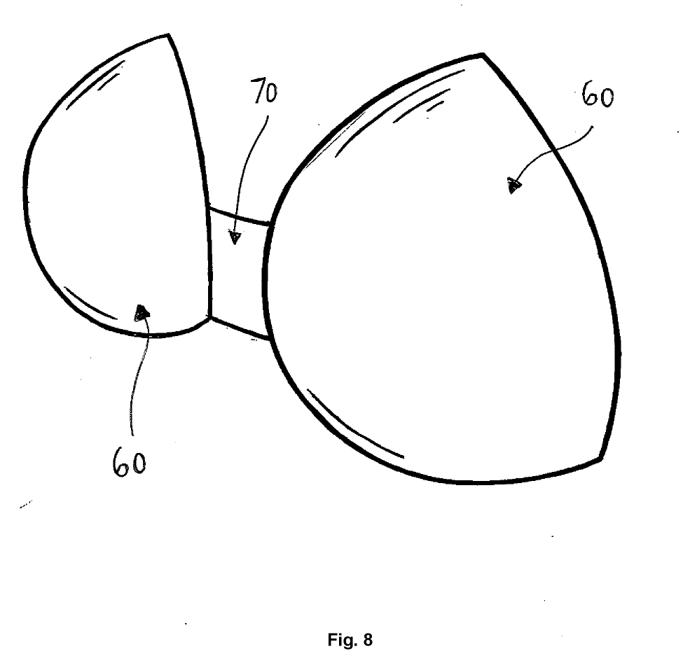

FIG. 9 is a front elevation view of a variation in shape of the padding component of the invention which will be sewn into the garment present invention thereof;

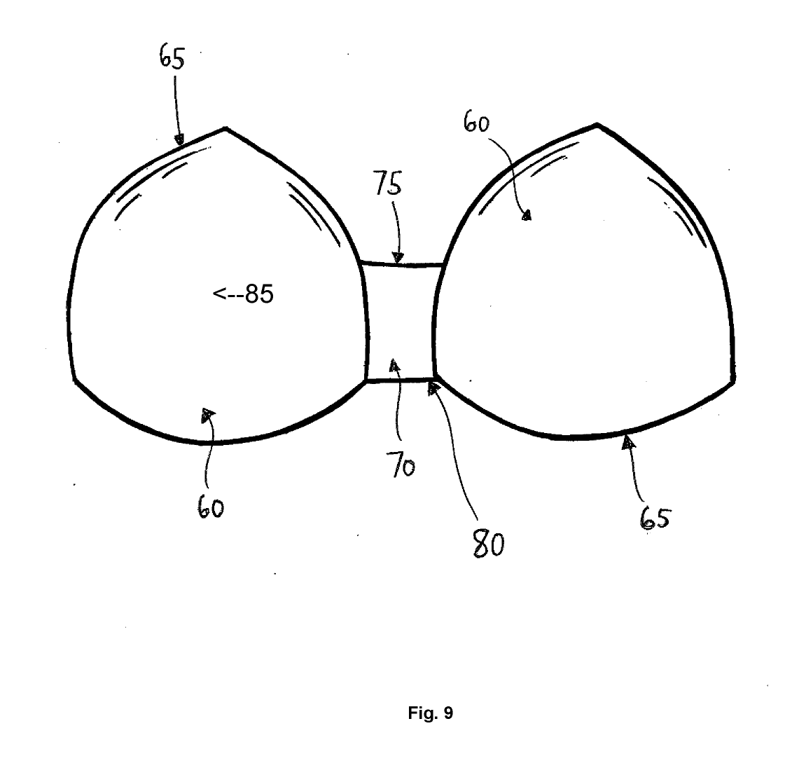

FIG. 10 is a front elevation view of a disassembled, alternative formation of the padding component of the invention consisting of two, separate breast mound prostheses and one piece of padded fabric stabilizer which are intended to be sewn together forming one piece, if alternately, one, fully developed piece of the enhanced and contoured foam padding with two, integral breast mound prostheses is not available to be manufactured for the garment present invention thereof;

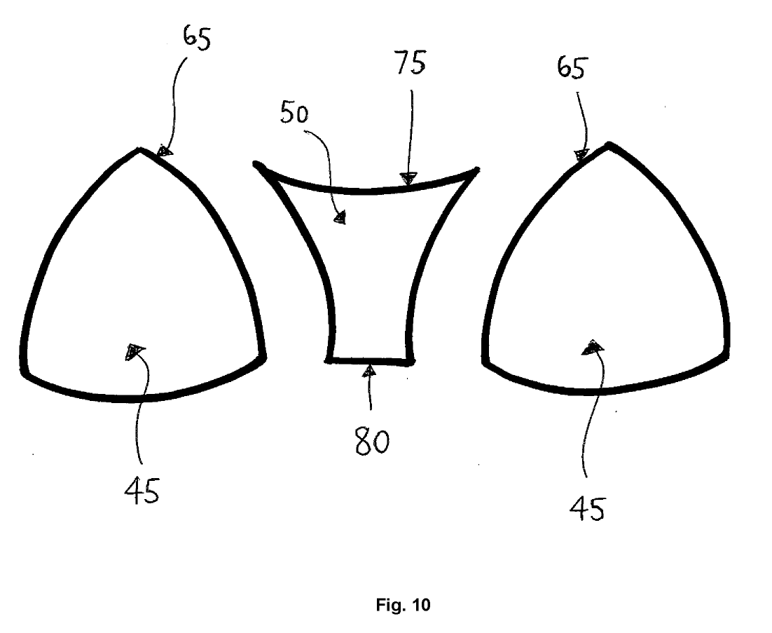

FIG. 11 is a front view of the garment present invention thereof;

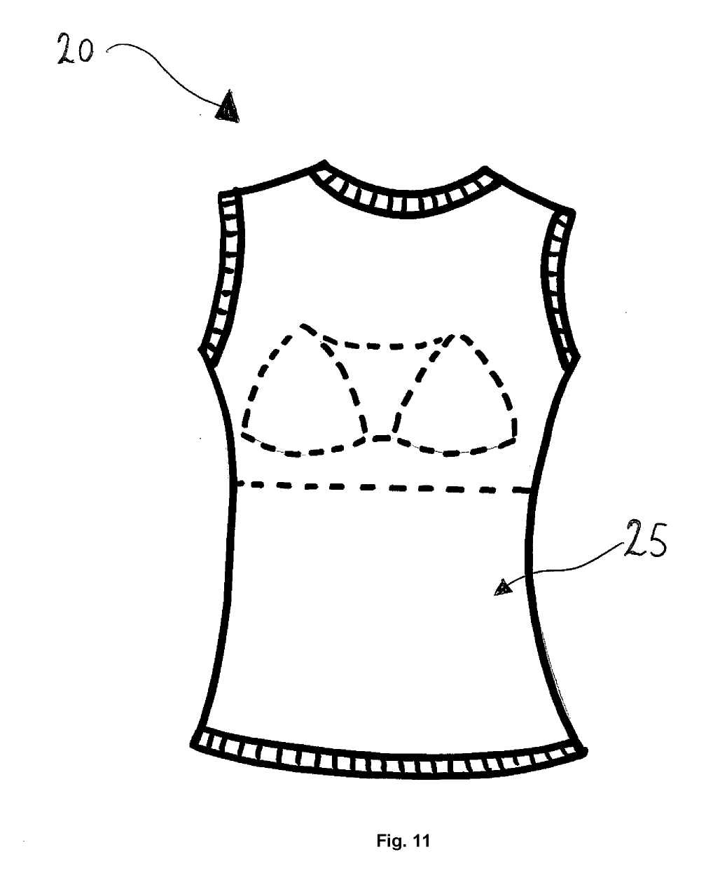

FIG. 12 is a front view of the padding component of the invention sewn onto an inner, middle layer of the garment present invention thereof;

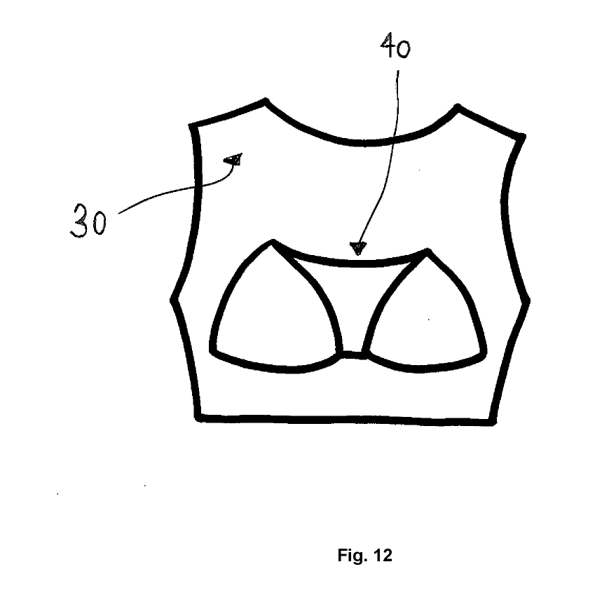

FIG. 13 is a front view of the garment present invention with the front layer of the said garment present invention removed thereof; and,

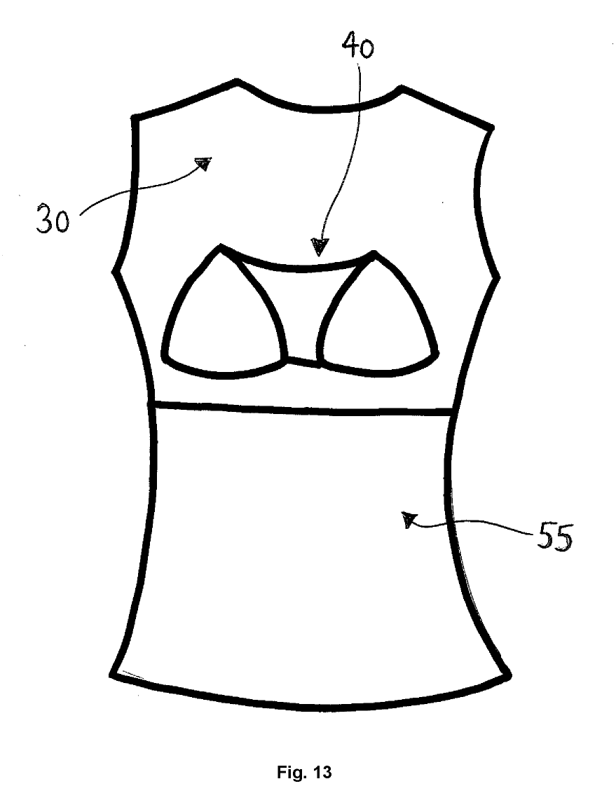

FIG. 14 is an illustration of the 3 layers of the garment present invention—front layer, middle layer with the single piece of enhanced and contoured foam padding with two, integral breast mound prostheses and the back layer of one of the representations of the garment present invention.

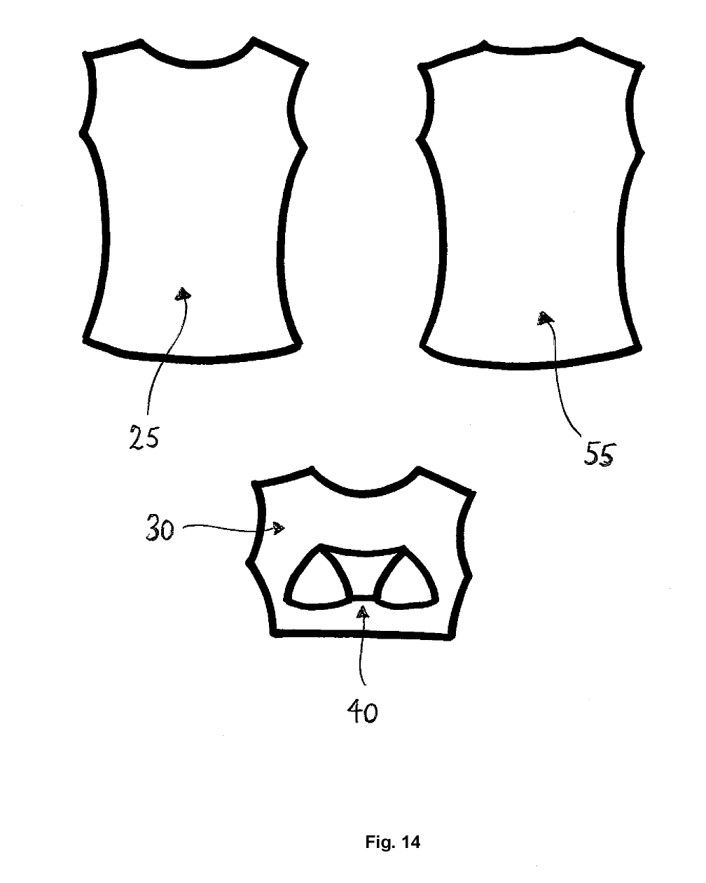

### DETAILED DESCRIPTION OF THE INVENTION

While this invention is invariable in its nature and essence that of being a garment with an enhanced and contoured lightweight foam padding with two, integral breast mound prostheses which are permanently sewn into this said garment for the mastectomy patient, this said garment invention is able to take on different shapes and sizes in order to properly accommodate any and all individuals wearing this invention who are seeking to restore or gain a natural looking breast mound appearance in a quick, convenient and comfortable fashion. The garment invention always includes a front layer, middle layer with a single piece of enhanced and contoured foam padding with two, integral breast mound prostheses and a back layer. All three layers are sewn together, appropriately, to form one garment present invention.

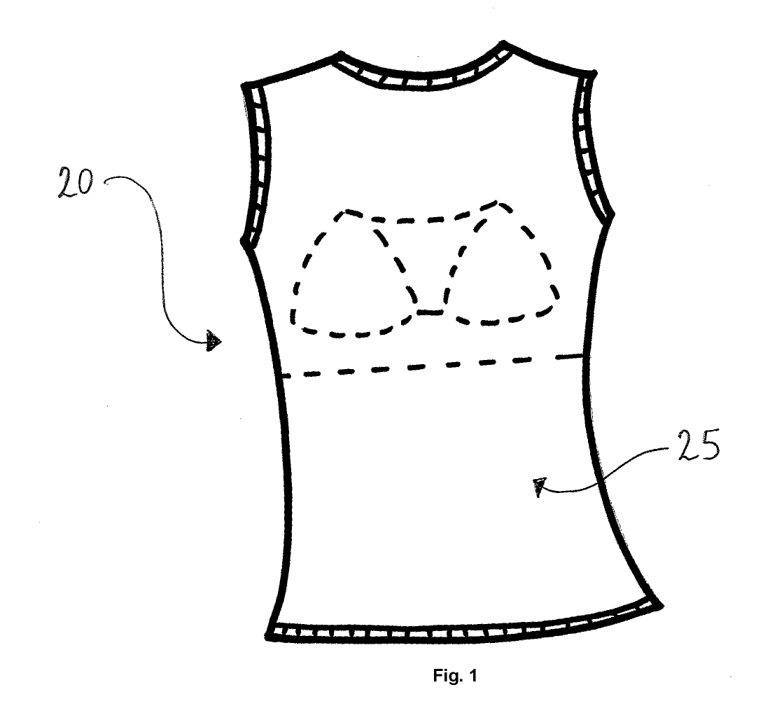

One representation of various styles of the said garment present invention is illustrated in FIG. 1. This representation in FIG. 1 includes a front layer 25, a middle layer 30 with a sewn on single piece of enhanced and contoured foam padding with two, integral breast mound prostheses 40 and a back layer 55. The front layer 25 can be of any style of a garment. The middle layer 30 is always a cropped piece of material which mimics the front layer 25 design and it is preferably made up of a soft 95%-100% cotton material to comfort the skin. The single piece of enhanced and contoured foam padding with two, integral breast mound prostheses 40 is always sewn onto the said middle layer 30 so that the front of the breast mound prostheses 60 or 45 on the padding 40 or 50 face the inside of the front layer 25 of the garment. This padding 40 consists of two, integral breast mound prostheses 60 or 45 which measure between ½″ to 1″ in thickness at the apex 85 of the breast mound prosthesis 60 or 45 and gradually tapers down in thickness towards the perimeter 65 of the breast mound prosthesis 60 or 45, measuring no more than ⅛″ in thickness. The mid-section 70 of the padding 40 measures between 3/16″ to ¼″ thick but the top perimeter 75 and bottom perimeter 80 of the mid-section 70 is no more than ⅛″ in thickness. The back layer 55 of the said garment present invention can also be of any style or design. The front layer 25, the middle layer 30 with padding 40 and back layer 55 are sewn together, appropriately to produce one representation of the said garment present invention. The breast mound prostheses are thicker than the typical bra cup pad making them more natural to the touch and more durable in keeping their shape. The mid section of the invention is foam padding making the invention helpful in protecting the sensitive mastectomy site. The mid section of the invention is either molded and created within a single piece of enhanced and contoured foam padding or an appropriate piece of foam fabric stabilizer is sewn between the two, separate breast mound prostheses to become one continuous piece of enhanced and contoured foam padding. While a full and complete description of the garment present invention has been announced in accordance with the patent statues, it should be acknowledged that modifications can be made without deviating from the essence and spirit of the following claims.

What is claimed is:

1. A garment containing lightweight, sternal padding with two integral breast mound prostheses for the bilateral mastectomy patient, or other, consisting of:
a front layer
a sternal padding with two breast mound prostheses
a middle layer and
a back layer.

2. The garment according to claim 1, wherein the front layer of the garment, the sternal padding with two breast mound prostheses, the middle layer of the garment and the back layer of the garment are joined together to form the garment.

3. The garment according to claim 1, wherein the sternal padding with two breast mound prostheses component of the said garment can be manufactured as one, continuous molded piece of padding with two breast mound prostheses.

4. The garment according to claim 1, wherein the sternal padding with two breast mound prostheses component of the said garment can be produced as one, continuous piece by adding two, separate breast mound prostheses to one piece of sternal padding.

5. The garment according to claim 1, wherein the sternal padding with two breast mound prostheses component of the said garment may be sewn within the said garment as three, separate parts—a piece of sternal padding and two breast mound prostheses on either side of the said sternal padding in order to offer additional options for producing the said garment while maintaining the essence of the sternal padding with two breast mound prostheses invention within the said garment.

6. The garment according to claim 1, wherein the sternal padding with two breast mound prostheses component is lightweight.

7. The garment according to claim 1, wherein the said garment is machine washable.

8. The garment according to claim 1, wherein the said garment is machine dryable.

9. The garment according to claim 1, wherein the said sternal padding with two breast mound prostheses component offers the wearer of the said garment two appropriately placed breast mound prostheses within their garment.

10. The garment according to claim 1, wherein the said garment is available in different sizes to accommodate every individual.

11. The garment according to claim 1, wherein the sternal padding with two breast mound prostheses component of the said garment is available in different sizes to accommodate every individual garment.

12. The garment according to claim 1, wherein the sternal padding with two breast mound prostheses component of the said garment is available in varying shapes to accommodate every individual garment.

13. The garment according to claim 1, wherein the said garment present invention is available in different styles of garments to accommodate different desired styles of garments.

14. The garment according to claim 1, wherein the essence of the sternal padding with two breast mound prostheses component remains constant in all styles of garments in that it offers the wearer of the said garment padded protection of the sensitive mastectomy site while exhibiting two breast mounds within their said worn garment.

15. The garment according to claim 1, wherein the said garment offers the convenience of wearing a garment with integral breast mound prostheses.

16. The garment according to claim 1, wherein the sternal padding with two breast mound prostheses component of the said garment is permanently sewn into the said garment.

17. The garment according to claim 1, wherein the said garment contains no broad band elastic.

18. The garment according to claim 1, wherein the said garment has no shelf bra.

19. The garment according to claim 1, wherein the thickness of the sternal padding component of the said garment may vary and be of any thickness in order to accommodate every individual wearing the garment present invention.

20. The garment according to claim 1, wherein the thickness of the breast mound prostheses component of the said garment may vary and be of any thickness in order to accommodate every individual wearing the garment present invention.

21. The garment according to claim 1, wherein no additional breast prostheses are needed within this said garment present invention to a have natural looking breast mound appearance while wearing this said garment present invention.

22. The garment according to claim 1, wherein the sternal padding with two breast mound prostheses component of the said garment is of a lightweight foam or similar material.

23. The garment according to claim 1, wherein the sternal padding with two breast mound prostheses resides within the garment and does not directly touch the skin of the individual wearing the said garment.

24. The garment according to claim 1, wherein the said garment offers the bilateral mastectomy patient an instant bilateral breast mound appearance when wearing the said garment present invention.

25. The garment according to claim 1, wherein the said garment present invention is unique in its attributes as a quick solution intended to help the bilateral mastectomy patient, or other, desiring to restore or gain a natural looking breast mound appearance in clothing—comfortably, safely and conveniently.
